(19) United States
(12) Patent Application Publication
(10) Pub. No.: US 2014/0064448 A1
(43) Pub. Date: Mar. 6, 2014

(54) COMMUNICATION METHOD AND RADIOGRAPHIC IMAGING SYSTEM AND APPARATUS

(71) Applicant: FUJIFILM Corporation, Tokyo (JP)

(72) Inventor: Takaaki ITO, Ashigarakami-gun (JP)

(73) Assignee: FUJIFILM Corporation, Tokyo (JP)

(21) Appl. No.: 14/012,893

(22) Filed: Aug. 28, 2013

(30) Foreign Application Priority Data
Aug. 31, 2012 (JP) 2012-191131

(51) Int. Cl. A61B 6/00

(52) U.S. Cl. CPC A61B 6/542
USPC 378/62; 378/97

(57) ABSTRACT

An X-ray imaging system includes an X-ray source for emitting X-rays. A radiation source driver drives the X-ray source. An X-ray imaging apparatus has an FPD device incorporated therein, has pixels arranged for storing signal charge according to X-rays transmitted through an object, for outputting a radiation image of the object by conversion in an electrical signal. An ionization chamber device is discrete from the X-ray imaging apparatus, for detecting X-rays transmitted through the object. An interface port is provided in the radiation source driver, for connection with the ionization chamber device. There is a communication path for connection between the interface port and the X-ray imaging apparatus, and for transmitting a sync signal, namely request signal S1 and enable signal S2, for operating the FPD device in synchronism with a start of emitting the X-rays.

### BACKGROUND OF THE INVENTION

1. Field of the Invention

The present invention relates to a communication method and a radiographic imaging system and apparatus. More particularly, the present invention relates to a communication method and a radiographic imaging system and apparatus, in which a radiation generating apparatus can be used in combination with a radiographic imaging apparatus with an FPD device without structural modification of the radiation generating apparatus.

2. Description Related to the Prior Art

An X-ray imaging system as a radiographic imaging system is well-known in the field of medical imaging. The X-ray imaging system includes an X-ray generating apparatus as a radiation generating apparatus, and an X-ray imaging apparatus as a radiographic imaging apparatus. The X-ray imaging apparatus forms an X-ray image from X-rays transmitted through a body of a patient. The X-ray generating apparatus includes an X-ray source as a radiation source, a radiation source driver and a source switch. The X-ray source applies X-rays to an object. The radiation source driver controls the X-ray source. The source switch is operable to input a command signal for starting the irradiation to the radiation source driver. The X-ray imaging apparatus includes an electronic cassette or a radiographic imaging unit, and a computer terminal or console unit. The radiographic imaging unit detects the X-ray image from X-rays transmitted through the body having various body parts. The computer terminal drives and controls the radiographic imaging unit, and stores and displays the X-ray image.

An FPD device (flat panel detector device) has been recently utilized as a radiographic imaging panel as a newly developed device in place of an X-ray film or imaging plate (IP). The FPD device has an imaging area in which arrays of pixels are arranged for storing signal charge according to dose of incident X-rays. The FPD device stores signal charge per pixels, reads the stored signal charge through switching elements such as TFTs, and electrically detects the X-ray image by conversion of the signal charge into voltage signal in a signal processor.

In the FPD device, the resetting is periodically carried out for the purpose of removing charge from the pixels to minimize influence of electric noise of dark current in the X-ray image, as a difference from the X-ray film or imaging plate. It is necessary to synchronize a start of the storing after the resetting with a start of irradiation of X-rays from the X-ray source.

JP-A 2011-041866 discloses synchronism in start in a radiographic imaging system in which a radiographic imaging apparatus has an FPD device. A network interface is provided, and couples a controllable radiation source driver to the radiographic imaging apparatus communicably. A single signal cable extends between the radiation source driver and the network interface. A communication system of a mixed use of wired and wireless manners is used for connection between the network interface and the radiographic imaging apparatus.

In FIG. 12, a radiographic imaging system with an FPD device of JP-A 2011-041866 is illustrated. For synchronism in the start, at first a controllable radiation source driver 200 sends a request signal S1 to an X-ray imaging apparatus 201 for checking whether irradiation of X-rays may be started. The X-ray imaging apparatus 201 terminates the resetting and starts the storing in response to a request signal S1, and sends an enable signal S2 to the radiation source driver 200 for authorization of the irradiation as a sync signal. The radiation source driver 200 starts irradiating X-rays in response to the enable signal S2. There is one communication path 202 through which the sync signal is sent and received between the radiation source driver 200 and the X-ray imaging apparatus 201.

In a medical facility having the radiographic imaging apparatus with an X-ray film or IP plate, introduction of the radiographic imaging apparatus with the FPD device may be planned. If a radiation generating apparatus for X-rays is exchanged for the specifics of the radiographic imaging apparatus with the FPD device, expense for the introduction will be considerably high. It is conceivable to reuse the radiation generating apparatus of the used type for the X-ray film or IP plate even in combination with the radiographic imaging apparatus. However, there occurs a problem in that no function for synchronism of the start between the radiographic imaging apparatus and the known type of the radiation generating apparatus. It is necessary to install or use circuit elements, interfaces, programs or the like for sending and receiving a request signal S1 or enable signal S2 properly for the purpose. Expense for the function of the synchronism will be additionally large.

### SUMMARY OF THE INVENTION

In view of the foregoing problems, an object of the present invention is to provide a communication method and a radiographic imaging system and apparatus, in which a radiation generating apparatus can be used in combination with a radiographic imaging apparatus with an FPD device without structural modification of the radiation generating apparatus.

In order to achieve the above and other objects and advantages of this invention, a communication method for a radiographic imaging system is provided, the radiographic imaging system including a radiation source for emitting radiation, a radiation source driver for driving the radiation source, and a radiographic imaging apparatus, having a radiographic imaging panel incorporated therein, having pixels arranged for storing signal charge according to the radiation transmitted through an object, for outputting a radiation image of the object by conversion in an electrical signal, wherein a first AEC signal output device is connectable to the radiation source driver, and is discrete from the radiographic imaging apparatus, for detecting a dose of the radiation transmitted through the object to output a first AEC signal for automatic exposure control. The communication method includes a step of carrying out connection in a communication path between the radiographic imaging apparatus and an interface port provided in the radiation source driver for connection with the first AEC signal output device, for transmitting a sync signal for operating the radiographic imaging panel in synchronism with a start of emitting the radiation.

The radiographic imaging apparatus communicates with the interface port with communication compatibility for transmission and reception in relation to a form of the sync signal.

In another preferred embodiment, there is communication incompatibility between the interface port and the radiographic imaging apparatus in relation to a form of the sync signal. The sync signal is converted by a signal converter into a form with communication compatibility.

The signal converter is provided in the radiographic imaging apparatus or in the communication path.

The radiographic imaging apparatus, when the sync signal is transmitted by the communication path with the radiation source driver, changes over the radiographic imaging panel from resetting of unnecessary signal charge of pixels to storing of the signal charge in the pixels.

The radiographic imaging apparatus includes a second AEC signal output device, discrete from the first AEC signal output device, for detecting a dose of the radiation transmitted through the object, to output a second AEC signal for automatic exposure control. The second AEC signal is transmitted by the communication path to the radiation source driver.

The radiation source driver shuts off irradiation of the radiation for the automatic exposure control according to the second AEC signal.

The second AEC signal output device includes a dose sensor for outputting a dose signal of the dose of the radiation.

The second AEC signal output by the second AEC signal output device is the dose signal. The radiation source driver includes a judging device for checking whether a cumulative dose of the radiation has reached a target dose according to the dose signal.

In one preferred embodiment, the second AEC signal output device is constituted by the dose sensor and a judging device for checking whether a cumulative dose of the radiation has reached a target dose according to the dose signal from the dose sensor. If reach of the cumulative dose to the target dose is detected by the judging device, a shut-off signal is output for the second AEC signal.

A second sync signal for the radiographic imaging panel in synchronism with shut-off of the radiation is transmitted through the communication path. The radiographic imaging apparatus, when the second sync signal is transmitted through the communication path with the radiation source driver, changes over the radiographic imaging panel from storing of signal charge in the pixels to reading of the signal charge from the pixels in a signal processing circuit.

The communication path is constituted by a signal line.

The signal line has a signal coupling device with first, second and third contact points. The first contact point is connected with the interface port. The second contact point is connected with the radiographic imaging apparatus to constitute the communication path in series with the first contact point. The third contact point is connected with the first AEC signal output device in a communicable manner with the interface port.

The signal coupling device includes a selector for changing over the second and third contact points and connecting a selected one of the second and third contact points with the first contact point.

In still another preferred embodiment, the sync signal is transmitted and received wirelessly in the communication path.

Also, a radiographic imaging system is provided, and includes a radiation source for emitting radiation. A radiation source driver drives the radiation source. A radiographic imaging apparatus has a radiographic imaging panel incorporated therein, has pixels arranged for storing signal charge according to the radiation transmitted through an object, for outputting a radiation image of the object by conversion in an electrical signal. An interface port is provided in the radiation source driver, for connection with a first AEC signal output device, the first AEC signal output device being discrete from the radiographic imaging apparatus, for detecting a dose of the radiation transmitted through the object to output a first AEC signal for automatic exposure control. There is a communication path for connection between the interface port and the radiographic imaging apparatus, and for transmitting a sync signal for operating the radiographic imaging panel in synchronism with a start of emitting the radiation.

There is communication incompatibility between the interface port and the radiographic imaging apparatus in relation to a form of the sync signal. Furthermore, a signal converter is provided in the communication path, for converting the sync signal into a form with communication compatibility between the interface port and the radiographic imaging apparatus.

Furthermore, a signal coupling device has first, second and third contact points. The first contact point is connected with the interface port. The second contact point is connected with the radiographic imaging apparatus to constitute the communication path in series with the first contact point. The third contact point is connected with the first AEC signal output device in a communicable manner with the interface port.

In another preferred embodiment, furthermore, first and second wireless interfaces are arranged along the communication path, for wirelessly transmitting the sync signal between.

Also, a radiographic imaging apparatus for a radiographic imaging system is provided, the radiographic imaging system including a radiation source for emitting radiation, a radiation source driver for driving the radiation source, and an interface port, provided in the radiation source driver, for connection with a first AEC signal output device for detecting a dose of the radiation transmitted through the object to output a first AEC signal for automatic exposure control. The radiographic imaging apparatus includes a radiographic imaging panel, discrete from the first AEC signal output device, having pixels arranged for storing signal charge according to the radiation transmitted through the object, for outputting a radiation image of the object by conversion in an electrical signal. There is a communication path for connection with the interface port, and for transmitting a sync signal for operating the radiographic imaging panel in synchronism with a start of emitting the radiation. A controller operates the radiographic imaging panel according to the sync signal.

Consequently, a radiation generating apparatus can be used in combination with a radiographic imaging apparatus with an FPD device without structural modification of the radiation generating apparatus, because the communication path can connect the radiographic imaging apparatus to the radiation source driver.

### BRIEF DESCRIPTION OF THE DRAWINGS

The above objects and advantages of the present invention will become more apparent from the following detailed description when read in connection with the accompanying drawings, in which:

FIG. 1 is an explanatory view illustrating an X-ray imaging system;

FIG. 2 is a block diagram schematically illustrating circuit elements in the X-ray imaging system;

FIG. 3 is a block diagram schematically illustrating a communication path;

FIG. 4 is a flowchart illustrating steps of X-ray imaging;

FIG. 5 is a block diagram schematically illustrating a preferred electronic cassette with an AEC function;

FIG. 6 is a block diagram schematically illustrating another preferred electronic cassette with a judging device for an AEC function;

FIG. 7 is a block diagram schematically illustrating another preferred communication path with a connector;

FIG. 8 is a block diagram schematically illustrating one preferred communication path with a selector;

FIG. 9 is a block diagram schematically illustrating another preferred communication path operating wirelessly;

FIG. 10 is a block diagram schematically illustrating still another preferred communication path with a wireless interface device;

FIG. 11 is a block diagram schematically illustrating another preferred communication path with a signal converter;

FIG. 12 is a block diagram schematically illustrating a communication path according to the prior art.

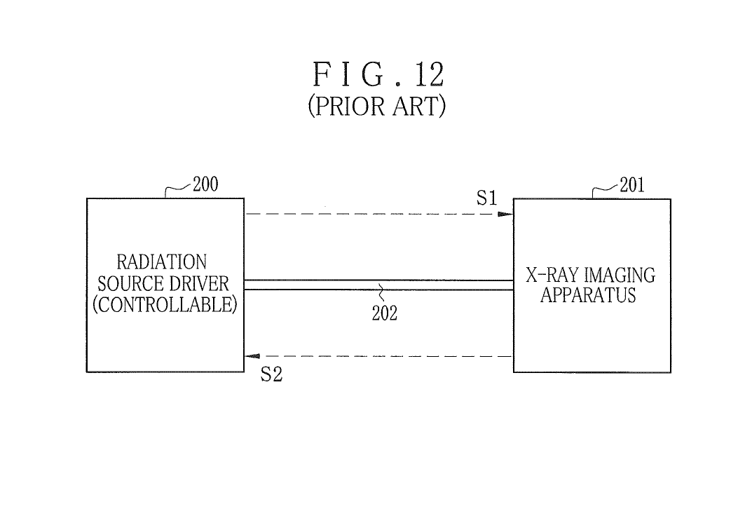

### DETAILED DESCRIPTION OF THE PREFERRED EMBODIMENT(S) OF THE PRESENT INVENTION

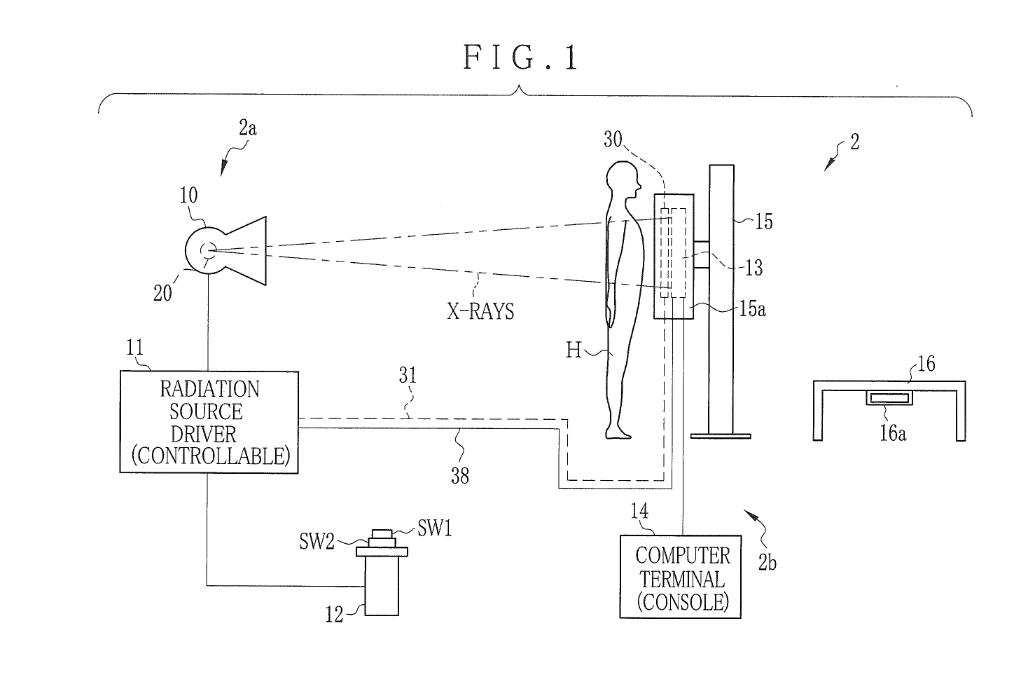

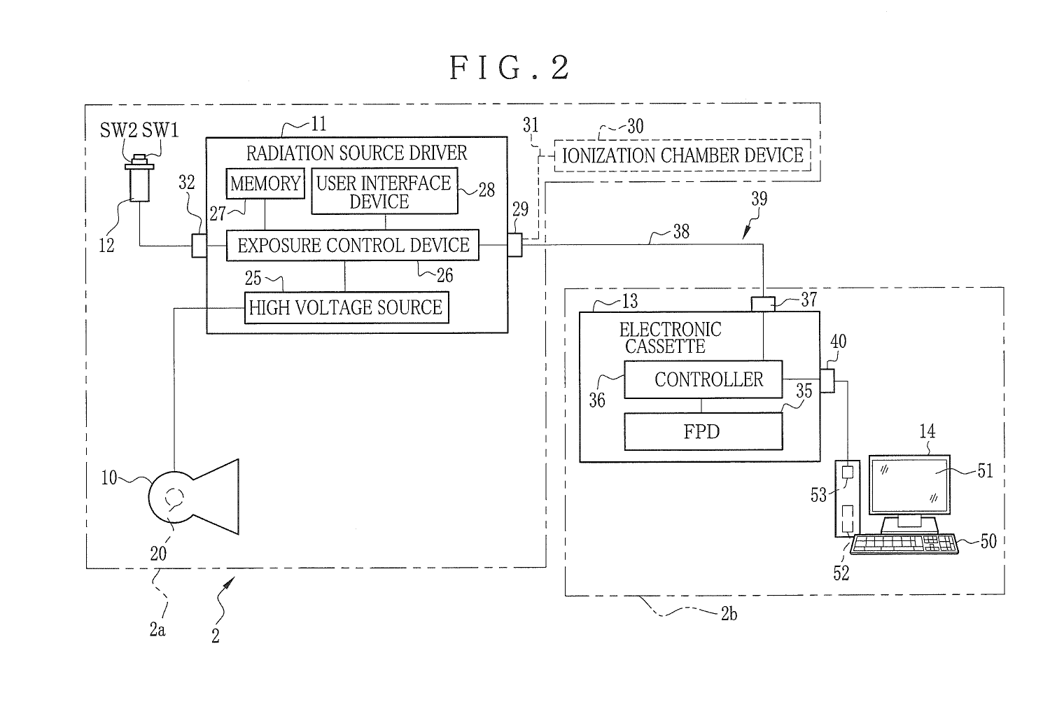

In FIGS. 1 and 2, an X-ray imaging system 2 as a radiographic imaging system is illustrated, and includes an X-ray source 10 as a radiation source, a (controllable) radiation source driver 11, a source switch 12, an electronic cassette 13 of a radiographic imaging panel, a computer terminal 14 (console unit), a floor stand 15 with an imaging station, and an imaging table 16 with an imaging station. The radiation source driver 11 controls the X-ray source 10. The source switch 12 is operable for starting warmup of the X-ray source 10 and starting irradiation of X-rays in the X-ray source 10. The electronic cassette 13 detects X-rays transmitted through the object H (patient) and outputs an X-ray image. The computer terminal 14 controls operation of the electronic cassette 13 and processes the X-ray image. The floor stand 15 is used for imaging the object H in an erect orientation. The imaging table 16 is used for imaging the object H in a horizontally lying orientation. An X-ray generating apparatus 2a as a radiation generating apparatus is constituted by the X-ray source 10, the radiation source driver 11 and the source switch 12. An X-ray imaging apparatus 2b as a radiographic imaging apparatus is constituted by the electronic cassette 13 and the computer terminal 14. The X-ray generating apparatus 2a is combinable with a film cassette and IP cassette in addition to the electronic cassette 13. Also, a moving mechanism (not shown) is disposed for setting the X-ray source 10 in a desired direction and position. The X-ray source 10 is used in both of the floor stand 15 and the imaging table 16.

The X-ray source 10 includes an X-ray tube 20 and a collimator (not shown) for limiting a region of irradiating X-rays from the X-ray tube 20. The X-ray tube 20 includes a cathode and an anode (target electrode). The cathode has a filament for emitting thermal electron. The anode receives collision of thermal electron from the cathode for emitting X-rays. The collimator includes four plates of lead, and a center opening. The four plates are radiopaque, and arranged in a form of a quadrilateral. The center opening is defined by the four plates for transmitting X-rays. The center opening is changeable for its size. Positions of the plates are shifted to determine a field of irradiation by changing the size of the center opening.

The radiation source driver 11 includes a high voltage source 25 and an exposure control device 26. The high voltage source 25 has a transformer for boosting an input voltage, generates a high voltage as tube voltage, and supplies the X-ray tube 20 with the tube voltage. The exposure control device 26 controls the tube voltage, a tube current and irradiation time of X-rays. The tube voltage determines radiation quality (energy spectrum) of X-rays from the X-ray source 10. The tube current determines an irradiation amount per unit time. The high voltage source 25 is connected to the X-ray source 10 by a high voltage cable.

A memory 27 stores information of the tube voltage, tube current, irradiation time and shut-off threshold in operation of the exposure control device 26, as imaging conditions predetermined for plural objects of interest in a human body. A user interface device 28 or input device, such as a touch panel or the like, is manually operated by an operator such as medical technician, radiologist and the like, to input imaging conditions. A count down timer (not shown) is incorporated in the exposure control device 26 for stopping irradiation of X-rays upon lapse of the predetermined irradiation time.

A target value of the irradiation time for the AEC is set sufficiently large for the purpose of preventing shortage of the dose because of termination of the irradiation before the instruction of terminating the irradiation in the AEC. It is possible to predetermine the maximum irradiation time defined in the X-ray source 10 for public regulation with safety. The exposure control device 26 controls the irradiation of X-rays according to the tube voltage, tube current, irradiation time and the like conditioned in the imaging condition. If it is judged in the AEC that cumulative dose of X-rays has reached an appropriate target dose, the AEC operates to shut off the irradiation of X-rays even if the irradiation time is equal to or shorter than the target irradiation time determined by the radiation source driver 11. In case of no AEC, irradiation time depending upon a body part of an object to be imaged is set. When the built-in count down timer detects lapse of the determined irradiation time, then the exposure control device 26 stops the irradiation.

The X-ray generating apparatus 2a includes an ionization chamber device 30, an interface port 29 (AEC interface) and a signal line 31 or cable. The ionization chamber device 30 is a first AEC signal output device (detection device) discrete from the electronic cassette 13. The signal line 31 connects the ionization chamber device 30 to the interface port 29. An example of the interface port 29 is a structure having a cable line and metal plates, the cable line extending from the signal line 31 by peeling cable jacket material, the metal plates squeezing the cable line for electrical connection. Note that the interface port 29 can be in a form of a press-fit connector or the like.

The ionization chamber device 30 is used for imaging with a film cassette, IP cassette or with an electronic cassette. Holders 15a and 16a are disposed on respectively the floor stand 15 and the imaging table 16. The ionization chamber device 30 is positioned on a front or rear surface of the electronic cassette at any one of the holders 15a and 16a. In FIG. 1, the ionization chamber device 30 is supported on the holder 15a of the floor stand 15. However, positioning of the ionization chamber device 30 is selectively determined for common use between the floor stand 15 and the imaging table 16, as the ionization chamber device 30 can be supported on the holder 16a of the imaging table 16. At the time of using the electronic cassette 13, the ionization chamber device 30 is disconnected and removed from the interface port 29 as indicated by the broken line.

Local areas (exposure areas) are disposed in the ionization chamber device 30 in a predetermined position, for example, the right or left lung (chest), the center of the abdomen, and the like. The ionization chamber device 30 outputs a voltage signal at a predetermined interval of sampling according to dose of X-rays transmitted to the local areas through the object H. The voltage signal is referred to as a first dose signal or first AEC signal.

The interface port 29 receives the first dose signal from the ionization chamber device 30 for entry to the exposure control device 26. Also, the interface port 29 sends a request signal S1, and receives an enable signal S2 from the ionization chamber device 30 as a response to the request signal S1 for starting the preparatory operation for detection in the ionization chamber device 30. In the present invention, the synchronous communication with the electronic cassette 13 is carried out by utilizing the function of the interface port 29.

The ionization chamber device 30 is a well-known device for use in combination with the film cassette or IP cassette. Thus, the radiation source driver 11 of almost all of the types has the interface port 29.

Specifically, a first port connector (not shown) is disposed at a distal end of the signal line 31 extending from the ionization chamber device 30. A second port connector (not shown) is disposed at a distal end of a signal line (38) extending from the X-ray imaging apparatus 2b. The interface port 29, when coupled connectively with the first port connector, is connected with the ionization chamber device 30, and when disengaged from the first port connector, is coupled connectively with the second port connector for connection with the X-ray imaging apparatus 2b.

A switch interface 32 is provided on the exposure control device 26. The source switch 12 is connected to the exposure control device 26 by the switch interface 32. An operator manipulates the source switch 12 to start irradiation of X-rays. The buttons SW1 and SW2 are structurally nested in the source switch 12, which is a two-step switch in which the button SW2 is depressible only after depressing the button SW1. When the source switch 12 is depressed halfway to turn on the button SW1, the exposure control device 26 generates a start signal for warmup of the X-ray tube 20. When the source switch 12 is depressed fully to turn on the button SW2, the exposure control device 26 generates the request signal S1 for checking allowance of starting irradiation of X-rays as a sync signal for start. The request signal S1 is output from the interface port 29 to the electronic cassette 13 or the ionization chamber device 30. The electronic cassette 13 or the ionization chamber device 30 generates the enable signal S2 as a response to the request signal S1. The exposure control device 26, upon receiving the enable signal S2 from the interface port 29, generates the drive signal. A start signal for warmup and the drive signal are sent to the high voltage source 25. The high voltage source 25 starts supplying the X-ray tube 20 with power for irradiation upon receiving the drive signal from the exposure control device 26.

For the AEC with the ionization chamber device 30 in combination with the film cassette or IP cassette, the exposure control device 26 checks whether the cumulative dose of X-rays has reached the target dose according to the first dose signal input to the interface port 29. The exposure control device 26 carries out accumulation of the first dose signal, compares the cumulative dose with the predetermined shut-off threshold or target dose, and judges the reach of the dose.

When the exposure control device 26 judges that the cumulative value becomes more than the shut-off threshold to detect the reach of the cumulative dose to the target dose, the exposure control device 26 stops supply of power from the high voltage source 25 to the X-ray tube 20 to shut off irradiation of X-rays. Note that if a level of the dose signal is remarkably low with influence of implant, it is possible to detect abnormality in the exposure control device 26 and stop irradiation of X-rays.

The exposure control device 26 shuts off irradiation of X-rays, and causes the interface port 29 to output a shut-off signal S3 for informing the shut-off of the irradiation. The ionization chamber device 30 stops sampling the first dose signal in response to the shut-off signal S3.

Also, the exposure control device 26 shuts off the irradiation of X-rays when the count down timer measures the irradiation time determined by the user interface device 28, and also when an input signal from the switch interface 32 discontinues upon termination of the full depression of the source switch 12.

The electronic cassette 13 includes an FPD device 35 (flat panel detector device) as a radiographic imaging panel, and a controller 36 for controlling the FPD device 35. The FPD device 35 includes a TFT active matrix substrate and plural arrays of pixels arranged thereon for storing charge according to the dose of X-rays transmitted through the object H. As is well-known, each of the pixels includes a photoelectric converter, a capacitor and a TFT as a switching element. The photoelectric converter generates charge (electron-hole pair) upon incidence of visible light. The capacitor stores charge generated by the photoelectric converter. The FPD device 35 reads signal charge stored in the photoelectric converters in each of the pixels for respectively the signal processing circuit through signal lines associated with respectively the arrays of the pixels. The FPD device 35 outputs an X-ray image by conversion into a voltage signal in the signal processing circuit. Note that the pixels may not have a capacitor.

The FPD device 35 includes a scintillator for converting X-rays to visible light, and is an indirect conversion type in which the visible light output by the scintillator is converted by pixels photoelectrically into a signal. Note that the scintillator and the TFT active matrix substrate may be a PSS type (penetration side sampling) in which X-rays enter the scintillator before the substrate, or an ISS type (irradiation side sampling) in which X-rays enter the substrate before the scintillator. Also, the scintillator may be omitted. An FPD device can be a direct conversion type in which X-rays are converted into electric charge by a conversion layer, for example, amorphous selenium layer, without a scintillator.

The controller 36 drives the TFT by use of the scan lines associated with the arrays of the pixels. The FPD device 35 is driven by the controller 36 to carry out storing, reading, and resetting. In the storing, the FPD device 35 stores signal charge for pixels according to dose of X-rays. In the reading, the FPD device 35 reads the stored signal charge from the pixels. In the resetting, the FPD device 35 removes the dark current charge created in the pixels. Also, the controller 36 processes image data of X-ray images from the FPD device 35 upon the reading in the image processing of such functions as offset correction, sensitivity correction, defect correction, and the like.

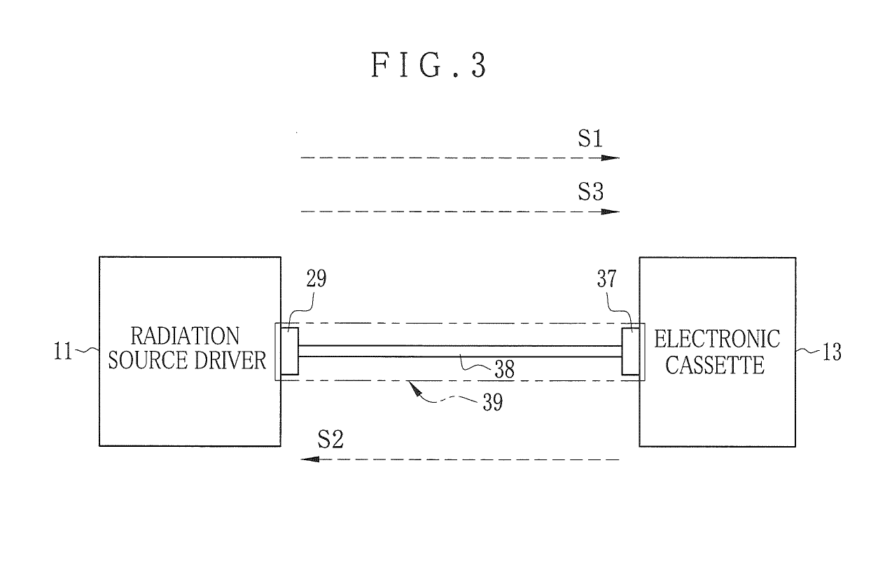

As illustrated in FIG. 3, a communication interface 37 is connected by a signal line 38 or cable to the interface port 29 of the radiation source driver 11. A communication path 39 is defined by the interface port 29, the communication interface 37 and the signal line 38. The communication interface 37 receives the request signal S1 generated by the interface port 29 for entry in the controller 36. The controller 36 upon receiving the request signal S1 changes over the FPD device 35 from the resetting to the storing to set the imaging mode instead of the standby mode. At the same time, the controller 36 outputs the enable signal S2 from the communication interface 37 to the interface port 29 as a sync signal. The enable signal S2 is in the same form as that output by the ionization chamber device 30. The communication interface 37 receives the shut-off signal S3 from the interface port 29 for entry in the controller 36. The controller 36 changes over the FPD device 35 from the storing to the reading in response to the shut-off signal S3 from the communication interface 37.

A communication interface 40 is on-line with the computer terminal 14 (console unit) in a wired manner or wirelessly for communication. The communication interface 40 sends and receives information including image data of X-ray images output by the FPD device 35 and an imaging condition determined by the computer terminal 14.

A portable housing of a box shape with a small thickness contains the FPD device 35 and the controller 36. In addition to the FPD device 35, the housing contains a battery (secondary cell) and an antenna. The battery supplies elements of the electronic cassette 13 with power at a predetermined voltage. The antenna wirelessly sends such data to the computer terminal 14 (console unit) as image data of X-ray images and the like.

The housing has a size according to the International Standards ISO 4090:2001 in a manner near to a film cassette and IP cassette. The electronic cassette 13 is removably set in each of the holders 15a and 16a of the floor stand 15 and the imaging table 16 of a conventional form for the film cassette and IP cassette to oppose the imaging area of the FPD device 35 to the X-ray source 10. According to one of the floor stand and the imaging table for use, the X-ray source 10 is moved by a source moving mechanism. Note that the electronic cassette 13 can be used discretely in a state placed on a table where the body H of the patient lies, or a state manually held by the patient. Note that the electronic cassette 13 can have a size not according to the International Standards ISO 4090:2001.

There is a user interface device 50 or input device which an operator manipulates for inputs, for example, a keyboard. The computer terminal 14 (console unit) controls the electronic cassette 13 according to the inputs. In the computer terminal 14, a display panel 51 displays X-ray images sent from the electronic cassette 13 by the communication interface 40. Data of the X-ray images are stored in a storage medium 52 in the computer terminal 14, or other storage devices such as a memory and storage server in connection with the computer terminal 14 by a network.

The computer terminal 14 (console unit) receives a medical request for examination with information of attributes, such as sex, age, and body part of the person as object H, and a purpose of imaging. The computer terminal 14 drives the display panel 51 to display the medical request. Various methods are available for inputting the medical request. For example, an external system for managing case information and condition information can input the medical request, for example, HIS (Hospital Information System) and RIS (Radiology Information System). Also, an operator may manually input the medical request. Examples of the body parts include a head, chest, abdomen, hands, fingers and the like. Also, information of viewing directions can be added to the body parts, such as front direction, lateral direction, diagonal direction, PA (posteroanterior direction) and AP (anteroposterior direction). An operator views various data in the medical request for examination on the display panel 51, and manipulates the user interface device 50 to input an imaging condition by referring to the items on the display panel 51.

Various imaging conditions are stored in the computer terminal 14 (console unit) for respectively body parts. Information of the imaging conditions includes data of a tube voltage, tube current and the like. The storage medium 52 stores the imaging conditions. According to a body part which an operator designates by use of the user interface device 50, one of the imaging conditions is read from the storage medium 52. A communication interface 53 is disposed so that the acquired imaging condition is sent through the communication interface 53 to the electronic cassette 13. For the radiation source driver 11, the operator refers to the imaging condition from the computer terminal 14 and manually inputs a similar imaging condition.

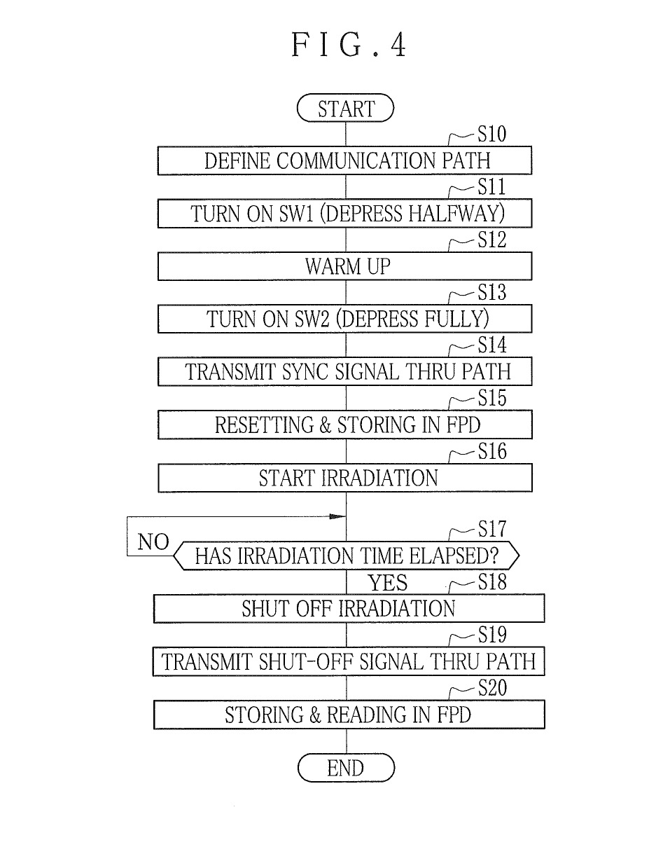

The operation of the X-ray imaging system 2 is described now by referring to FIG. 4 for one sequence of imaging with X-rays.

At first, the interface port 29 of the radiation source driver 11 is connected to the communication interface 37 of the electronic cassette 13 by the signal line 38 for defining the communication path 39 in the step S10.

After the preparation, the object H is positioned on one of the floor stand 15 and the imaging table 16. A height and horizontal position of the electronic cassette 13 are adjusted to target a region of interest in the object H. Then a height, horizontal position and field size of the X-ray source 10 are adjusted according to the position and a size of the region of interest of the electronic cassette 13. An imaging condition for the radiation source driver 11 and the computer terminal 14 (console unit) is determined. Information of the imaging condition from the computer terminal 14 is input to the electronic cassette 13.

When the imaging is ready, the operator depresses the source switch 12 halfway (turns on the switch SW1) in the step S11. The radiation source driver 11 sends a start signal to the high voltage source 25 for warmup. The high voltage source 25 starts supplying the X-ray tube 20 with power for its warmup in the step S12.

The operator depresses the source switch 12 halfway and then depresses the source switch 12 fully to turn on the switch SW2 (S13) by manually monitoring time required for the warmup. Then communication through the communication path 39 is carried out between the radiation source driver 11 and the electronic cassette for transmitting a request signal S1 for starting the irradiation and an enable signal S2 for the irradiation in the step S14. In the electronic cassette 13, the operation of the FPD device 35 is changed over from the resetting to the storing in the step S15.

The enable signal S2 generated by the electronic cassette 13 is input to the radiation source driver 11. Thus, the X-ray tube 20 emits X-rays in the step S16.

The radiation source driver 11 starts measuring time by setting the irradiation time in the count down timer according to the imaging condition at the same time as the start of the irradiation. When the predetermined irradiation time is measured by the count down timer (yes in the step S17), the high voltage source 25 is stopped by the radiation source driver 11 from supplying power to the X-ray tube 20. Irradiation of the X-ray source 10 is shut off (S18). Also, a shut-off signal S3 for the irradiation is output to the electronic cassette 13 through the communication path 39 (S19).

The shut-off signal S3 from the radiation source driver 11 is input to the electronic cassette 13. The FPD device 35 is changed over from the storing to the reading, and outputs image data of X-ray images in the step S20. After the reading, the FPD device 35 returns to the standby mode for resetting. The image data from the FPD device 35 is processed in image processing in various functions, and sent to the computer terminal 14 for the display panel 51 to display the X-ray images for the purpose of medical diagnosis. Thus, the X-ray imaging of one sequence is terminated.

For imaging with the film cassette or IP cassette, the ionization chamber device 30 is connected to the interface port 29. The AEC is carried out in the exposure control device 26 according to the first dose signal from the ionization chamber device 30. At the time of operation with AEC, irradiation of X-rays is shut off when the count down timer measures the irradiation time determined by the user interface device 28.

The interface port 29 is provided in the radiation source driver 11 of any one of known types as described above. The radiation source driver 11 can send the request signal S1 to and receive the enable signal S2 from the ionization chamber device 30 with the interface port 29. The electronic cassette 13 can be used without structurally modifying the radiation source driver 11 because the communication path 39 on the basis of the interface port 29 transmits the request signal S1 and the enable signal S2 with the electronic cassette 13. Among various medical service providers, an obstacle to introduction of the electronic cassette 13 in hospitals can be reduced effectively, so that marketing of the electronic cassette 13 can be enhanced. Also, the film cassette, IP cassette and the like can be used in the manner of the known techniques.

A grid may be used in the X-ray imaging system 2 for removing stripes of scatter created while X-rays pass the object H. The grid is a plate having a small thickness and including radio-transparent and radiopaque layers extending in a first direction of the pixels of the FPD device and arranged alternately in a second direction of the pixels. The grid is inserted between the object H and the electronic cassette and opposed to an incident surface of the electronic cassette.

In the X-ray imaging system 2 with the grid, a bucky device or holder can be disposed on the imaging table or floor stand for moving the grid between the start and end of the irradiation, so as to set stripes of the grid inconspicuous with radio-transparent and radiopaque layers. In compliance with this, a signal interface device may be incorporated in the radiation source driver for synchronism between the bucky device and irradiation of X-rays for the start and end. Also, it is possible to connect the electronic cassette to the signal interface device for the purpose of synchronizing the start of the changeover of the FPD device 35 from the resetting to the storing, or synchronizing its changeover from the storing to the reading.

However, a sync signal output by the signal interface device for the bucky device has signal components of a complicated combination, and must be read correctly on the side of the electronic cassette, because the sync signal has a signal component of a condition of driving the bucky device, such as a moving speed of the grid. Differences of models of the bucky device are considerably large due to the manufacturers. It is necessary in the electronic cassette to compensate for differences in the sync signal which is output by the signal interface device for the bucky device. In contrast with this, a sync signal from the interface port 29 is has signal components of a comparatively simple combination. The ionization chamber device 30 is well-known. Almost all of the known types of the radiation source driver 11 have the interface port 29. Differences of the sync signal between the manufacturers are rather small. Accordingly, it is more preferable to read a sync signal from the interface port 29 than to read this from the signal interface device for the bucky device, because of compatibility with the X-ray generating apparatus 2a of any one of various manufacturers.

In the first embodiment, the communication path 39 with the interface port 29 is utilized for synchronous communication between the radiation source driver 11 and the electronic cassette 13 for start. However, it is possible to start in synchronous communication according to a command signal from the source switch 12. A signal interface device is disposed between the source switch 12 and the switch interface 32. The electronic cassette 13 is connected with the signal interface device. When a command signal of the full depression is input by the source switch 12, the request signal S1 is sent by the signal interface device to the electronic cassette 13. When the enable signal S2 is input to the signal interface device by the electronic cassette 13, an auxiliary signal is input by the signal interface device to the switch interface 32 correspondingly to the command signal of the full depression of the source switch 12. Note that there is a drawback of a higher cost with the signal interface device. Thus, it is preferable that the communication path 39 with the interface port 29 is used to start in synchronous communication.

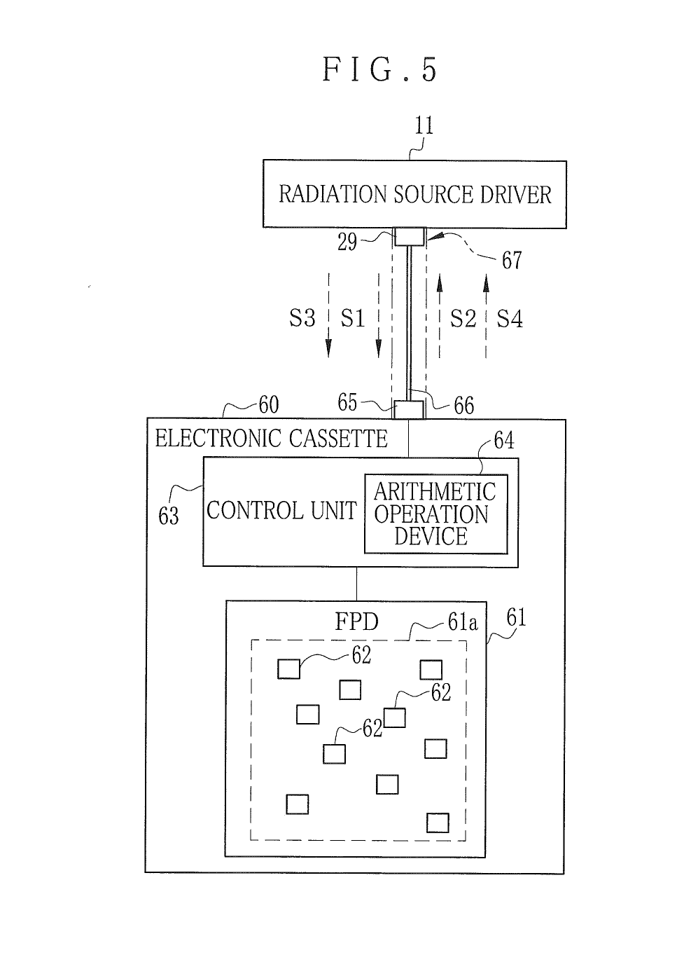

In the above embodiment, the electronic cassette 13 does not have the AEC function. In contrast, another preferred electronic cassette 60 illustrated in FIG. 5 has an AEC function.

In FIG. 5, the electronic cassette 60 includes an FPD device 61 (flat panel detector device) and a control unit 63. The electronic cassette 13 is repeated but with differences in that dose sensors 62, an arithmetic operation device 64 and a communication interface 65 are provided. The dose sensors 62 are disposed on an imaging area 61a of the FPD device 61 for detecting a dose of X-rays. The arithmetic operation device 64 is incorporated in the control unit 63. The communication interface 65 has functions of an interface for communication to the radiation source driver 11 with the sync signal and an interface port (AEC interface) for communication to the radiation source driver 11 with the second AEC signal. Elements similar to those of the above embodiment are designated with identical reference numerals. In the embodiment, the dose sensors 62 and the control unit 63 constitute the second AEC signal output device, which outputs a second dose signal S4 to the radiation source driver 11 as a second AEC signal, hereafter to be described.

A storage medium in the computer terminal 14 stores information of local areas (exposure areas) of the dose sensors 62 in addition to information of the tube voltage and tube current. The information of the local areas is read in the electronic cassette 60 at the time of setting the imaging condition.

The dose sensors 62 detect dose of X-rays to the imaging area 61a of the FPD device 61 having the pixels. The arithmetic operation device 64 in the control unit 63 is supplied with a second dose signal S4 output by the dose sensors 62. The dose sensors 62 are arranged regularly without local unevenness in the imaging area 61a.

The dose sensors 62 are constituted by part of the pixels. The particular pixels as the dose sensors 62 are ready to acquire a second dose signal S4 according to the generated charge even while the pixels for the imaging are in the course of the storing. An example of the dose sensors 62 is pixels in which a source and a drain of TFTs are short-circuited to one another. Another example of the dose sensors 62 is pixels in which a photoelectric conversion section is directly connected to a signal line without a TFT, for flow of the generated charge to a signal processing circuit irrespective of turning on and off of a TFT. A still another example of the dose sensors 62 is pixels of which a TFT is driven discretely from the TFTs for the pixels of the imaging.

Also, it is possible to detect dose of X-rays by monitoring a current through a bias line connected with particular pixels, as it is possible to utilize a current according to charge generated with pixels on the bias line for supplying bias voltage in the FPD device. In this structure, the pixels for monitoring the current through the bias lines are dose sensors. Furthermore, the dose can be detected by monitoring a leak current flowing from pixels. In this structure, the pixels for monitoring the leak current are dose sensors. Also, dose sensors can be discretely disposed in an imaging area in a structure different from the pixels for discrete outputs.

The arithmetic operation device 64 starts sampling the second dose signal S4 when the FPD device 61 changes over from a standby mode of the repeated resetting to an imaging mode for starting the storing. At each time that the second dose signal S4 is sampled, the arithmetic operation device 64 arithmetically determines an average (maximum, mode value, sum or the like) of the second dose signal S4 from the dose sensors 62 positioned in a second local area according to the body part.

The arithmetic operation device 64 calibrates the second dose signal S4 to a level of the first dose signal output by the ionization chamber device 30, so that the second dose signal S4 is receivable in the interface port 29. Specifically, the arithmetic operation device 64 multiplies the second dose signal S4 by a coefficient which is according to output levels of the first and second (S4) dose signals in irradiation of X-rays in the absence of the object H. For example, let 1 be the output level of the first dose signal in the absence of the object H. Let 10 be the output level of the second dose signal S4 in the absence of the object H. The second dose signal S4 is multiplied by 0.1. It is possible arithmetically to determine a coefficient for multiplication of the second dose signal S4 according to such parameters as sensitivity of the ionization chamber device 30 and the dose sensors 62 to X-rays, distances between the X-ray source 10 and the ionization chamber device 30 and between the X-ray source 10 and the dose sensors 62 (the imaging area 61a of the FPD device 61).

A signal line 66 or cable connects the communication interface 65 to the interface port 29 of the radiation source driver 11. A communication path 67 is defined by the interface port 29, the communication interface 65 and the signal line 66. The communication interface 65, in addition to transmission of the request signal S1, the enable signal S2 and the shut-off signal S3, sends the second dose signal S4 to the interface port 29 after calibration in the arithmetic operation device 64 as an AEC signal. In a manner similar to the first dose signal of the first embodiment, the exposure control device 26 of the radiation source driver 11 checks whether the cumulative dose of X-rays to the imaging area 61a has reached the target dose according to the cumulative value of the second dose signal S4 to the interface port 29. For the succeeding steps, the first embodiment is repeated. The electronic cassette 60 with the function of the AEC can be used without modifying the radiation source driver 11, because the AEC signal is transmitted through the communication path 67 defined by cooperation of the interface port 29 in addition to the sync signal.

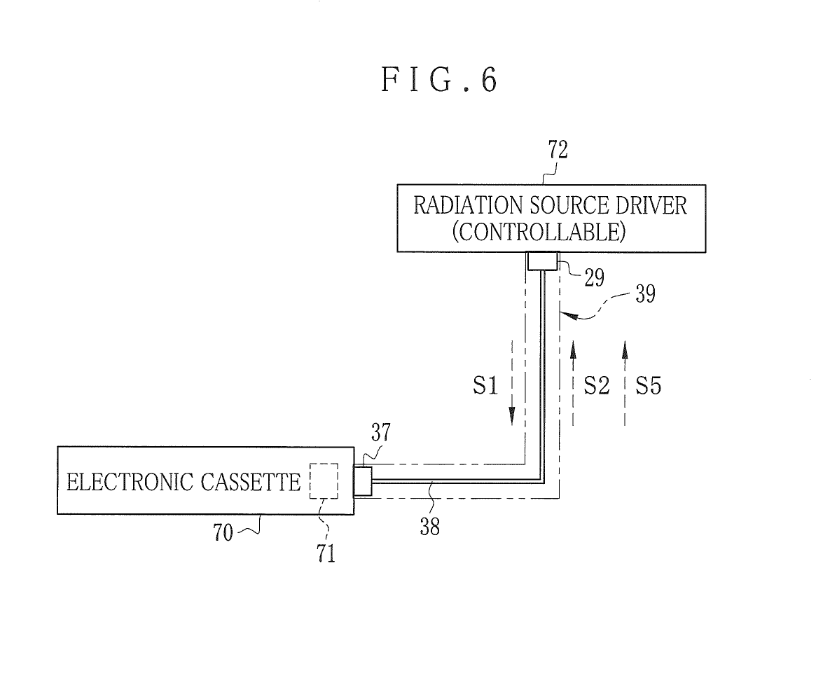

In the embodiment, a reach of the cumulative dose of X-rays to the target dose is detected in the radiation source driver 11. In FIG. 6, another preferred electronic cassette 70 has a function of detecting a reach of the cumulative dose of X-rays to the target dose. A judging device 71 in the electronic cassette 70 reads the cumulative value of the second dose signal S4, and outputs a shut-off signal S5 if the cumulative dose of X-rays reaches the target dose. A (controllable) radiation source driver 72 is supplied with the shut-off signal S5 through the communication interface 37. In response, the radiation source driver 72 shuts off the irradiation of X-rays of the X-ray source 10. In the embodiment of FIG. 6, the second AEC signal output device includes the judging device 71 in addition to the dose sensors 62 and the control unit 63 of FIG. 5.

In a certain variant example, the electronic cassette is constructed to output a shut-off signal as an AEC signal. The radiation source driver receives the dose signal as an AEC signal but does not receive the shut-off signal. To this end, the electronic cassette 70 of FIG. 6 is modified as follows. For this example, the electronic cassette 70 continues outputting a dose signal of dummy data to the radiation source driver until the cumulative dose is found to reach the target dose in the judging device 71 in the electronic cassette 70. When it is judged in the judging device 71 that the cumulative dose has reached the target dose, the electronic cassette outputs a signal to the radiation source driver at a level corresponding to a dose signal according to detection in the judging device for the reach of the cumulative dose to the target dose.

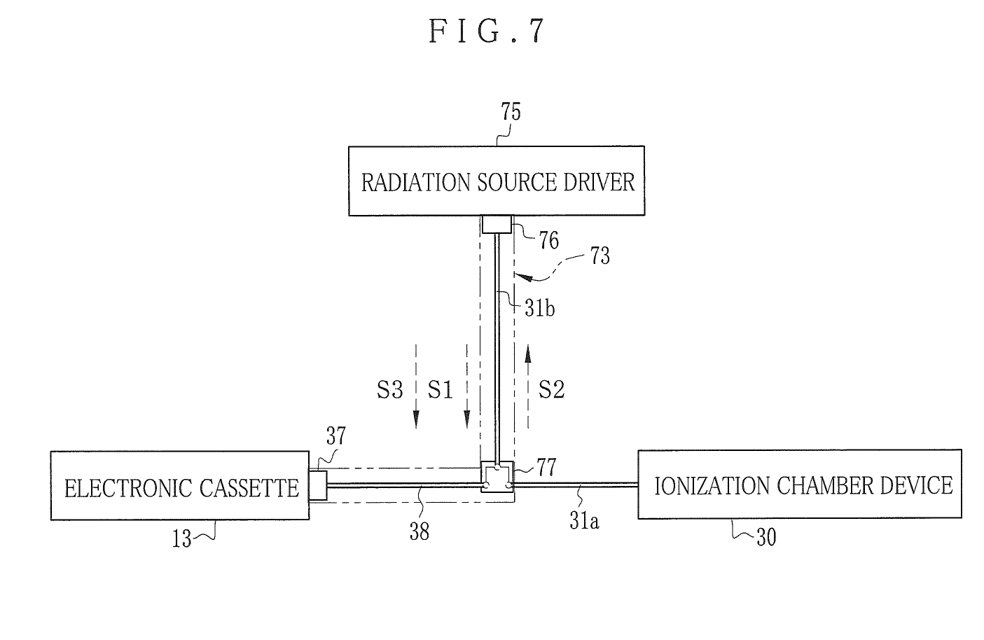

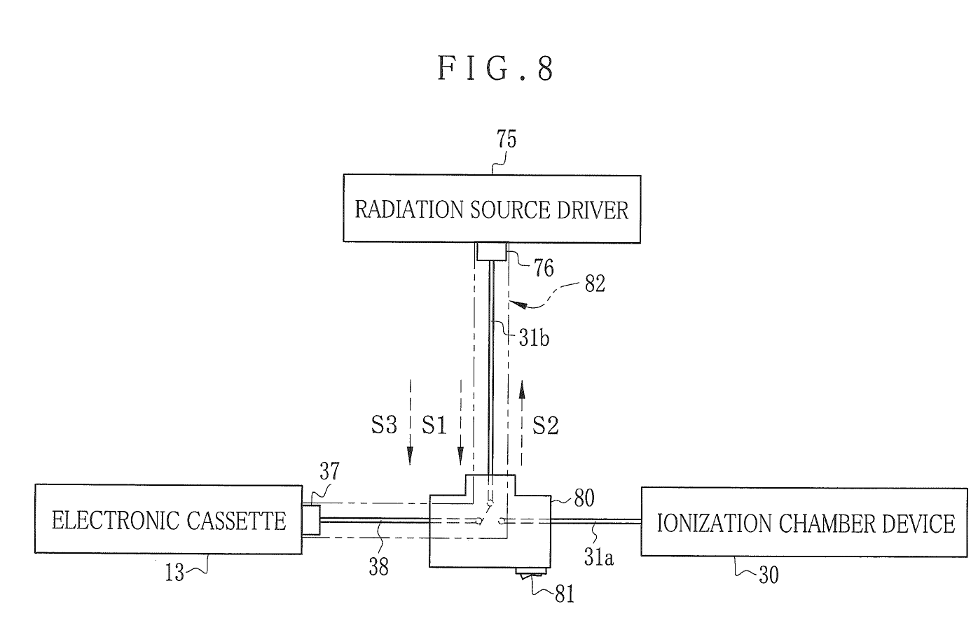

In the above embodiments, the ionization chamber device 30 is removable from the radiation source driver 11, 72. In contrast, FIGS. 7 and 8 illustrate a preferred (controllable) radiation source driver 75 where the ionization chamber device 30 is disposed unremovably.

In FIGS. 7 and 8, the radiation source driver 11 is repeated in the radiation source driver 75 but with a difference in that an interface port 76 (AEC interface) receives the signal line 31. The signal line 31 is unremovable from the ionization chamber device 30 and the interface port 76 in a wired manner without separation. Elements similar to those of the above embodiments are designated with identical reference numerals.

In FIG. 7, the signal line 31 is constituted by two signal lines 31a and 31b or cables. There is a signal coupling device 77 or terminal block connector with contact points to which the signal line 31a of the ionization chamber device 30 and the signal line 38 are connected. The signal line 31b of the interface port 76 is connected to a contact point of the signal coupling device 77. Thus, a communication path 73 is defined by connection of the signal lines 31 and 38. Note that crosstalk may occur with two of the enable signal S2 in the interface port 76 to cause an error in the radiation source driver 75 because each of the electronic cassette 13 and the ionization chamber device 30 may receive the request signal S1. It is necessary to turn off the power source for an unnecessary one of the electronic cassette 13 and the ionization chamber device 30, or to disconnect one of the signal cables from the signal coupling device 77 in association with the unnecessary device.

In FIG. 8, a selector 80 is provided in place of the signal coupling device 77, and operates between the signal lines 31a, 31b and 38. A selection switch 81 is disposed on the outside of a housing of the X-ray generating apparatus 2a, and changes over the contact point of the signal line 31b between the signal lines 31a and 38 selectively. In the case of using the electronic cassette 13, the selector 80 selects the contact point of the signal line 38. A communication path 82 is defined by changeover of the selector 80 for transmitting the sync signal. In the case of using the ionization chamber device 30, the selector 80 selects the contact point of the signal line 31a to transmit the sync signal and the first dose signal between the radiation source driver 75 and the ionization chamber device 30. Thus, erroneous detection in the radiation source driver 75 can be prevented, because of preventing two of the enable signal S2 from simultaneous entry in the radiation source driver 75 in a manner distinct from the structure of FIG. 7.

Note that the signal line 31b can have a small length. Also, it is possible for the signal coupling device 77 or the selector 80 directly to contact the interface port 76 without using the signal line 31b.

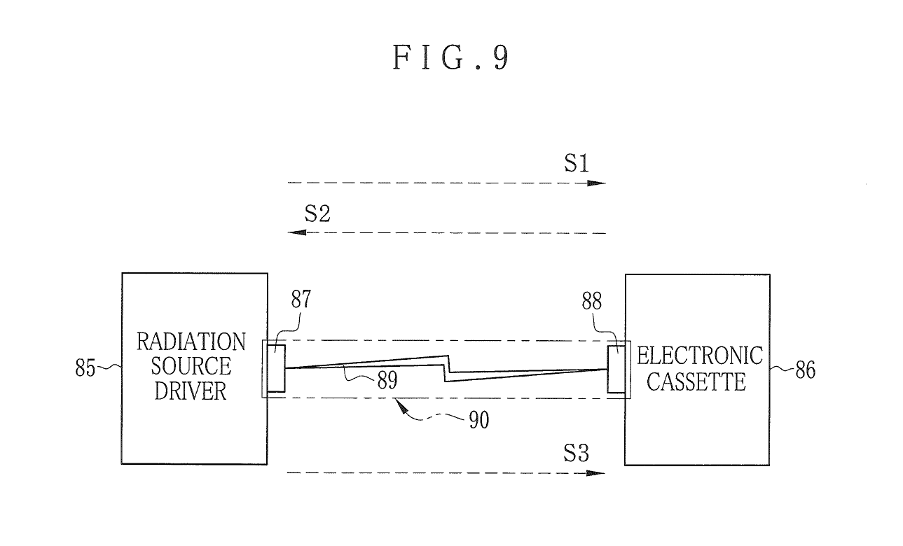

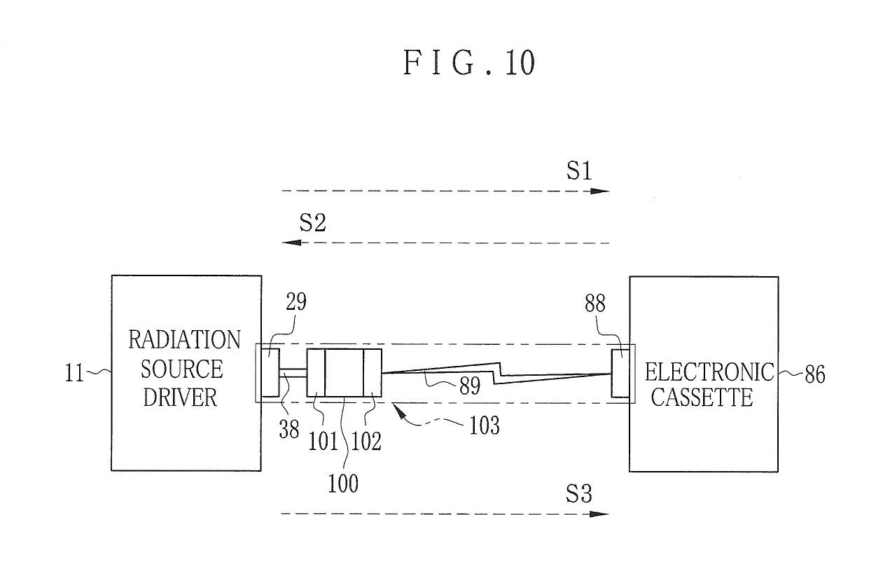

In the above embodiments, the communication path is defined in a wired manner. In contrast, FIGS. 9 and 10 illustrate preferred communication paths including at least one part in a wireless manner.

In FIG. 9, a (controllable) radiation source driver 85 includes a wireless interface port 87 with a communication antenna (AEC interface). An electronic cassette 86 includes a wireless interface 88 with a communication antenna for wireless communication with the wireless interface port 87. A radio wave 89 travels between the wireless interface port 87 and the wireless interface 88 wirelessly to transmit the request signal S1, the enable signal S2 and the shut-off signal S3, in the same manner as the path between the interface port 29 and the communication interface 37 in the first embodiment. A communication path 90 is defined by the wireless interface port 87, the wireless interface 88 and the radio wave 89.

In FIG. 10, a wireless interface device 100 or signal interface device is disposed between the radiation source driver 11 and the electronic cassette 86. The wireless interface device 100 has a first connection port 101 for coupling to the interface port 29 of the radiation source driver 11 with the signal line 38, and a wireless second connection port 102 with a communication antenna for coupling to the wireless interface 88 of the electronic cassette 86 with the radio wave 89. The wireless interface device 100 is positioned near to the radiation source driver 11, so a communication path 103 is constituted mainly by a path of the radio wave 89. The communication path 103 is defined by the interface port 29, the wireless interface 88, the connection ports 101 and 102, the signal line 38 and the radio wave 89.

Therefore, degree of freedom in the disposition of the various elements in the X-ray generating apparatus 2a can be high without considering extension of cables, because of the use of the wireless transmission. Handlability of the electronic cassette 86 can be high typically when the electronic cassette 86 is held manually by the patient or object H, or used separately, because of the wireless structure. It is possible in FIG. 10 to use the electronic cassette 86 of the wireless structure in the radiation source driver 11 without a wireless structure, as the wireless interface device 100 is utilized. Note that a wireless system other than the radio system can be used, for example, infrared transmission system.

Widely available interface ports for the radiation source driver are standardized in relation to the sync signal. However, there remain interface ports with a difference in the standard of the sync signal. It is necessary to convert the request signal S1 in a form receivable in the electronic cassette, or convert the enable signal S2 in a form receivable in a radiation source driver.

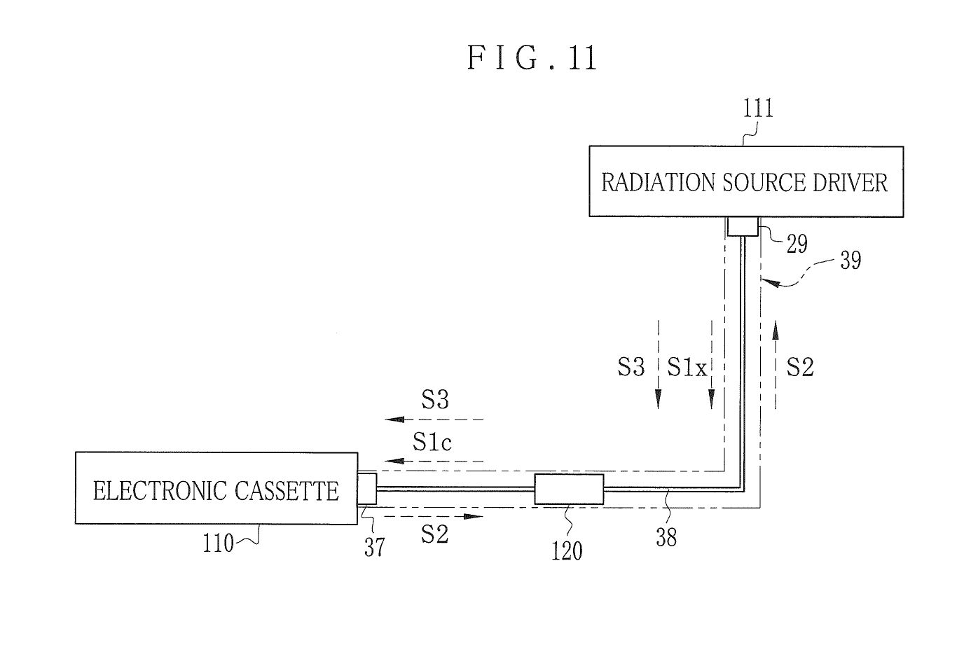

In FIG. 11, a preferred embodiment with an electronic cassette 110 and a (controllable) radiation source driver 111 is illustrated. The radiation source driver 111 outputs a request signal S1x of +5 V as a high level signal. The electronic cassette 110 is so constructed that a request signal S1c of −10 V as a low level signal is receivable. For this difference in the signal form, a signal converter 120 is provided in the signal line 38 for converting the request signal Six to the request signal Sic. Thus, the electronic cassette 110 can be used without modifying the radiation source driver 111. Furthermore, the signal converter 120 may be incorporated in the electronic cassette 110. Also, a signal form of the enable signal S2 or shut-off signal S3 may be different between the radiation source driver and the electronic cassette. For such a construction, the signal form is converted in the manner of the signal converter 120 for the request signal S1.

Furthermore, two or more of the structures of the above embodiments of the invention may be combined together. For example, the electronic cassette with the AEC function of FIGS. 5 and 6 (second embodiment) can be replaced with the electronic cassette of any one of the various embodiments.

In the above embodiments, the computer terminal 14 (console unit) is separate from the electronic cassette 13. However, the electronic cassette 13 may include a section of the computer terminal 14 as a unified component. Also, the radiation source driver 11 can include a section of the computer terminal 14 as one component. Furthermore, an additional control unit for imaging can be connected between the electronic cassette 13 and the computer terminal 14.

In the above embodiments, the ionization chamber device 30 is the first AEC signal output device. However, the first AEC signal output device may be other suitable devices, such as an FPD device with photo diodes (TFTs) and scintillators for detecting a dose.

In the above embodiments, the FPD device 35 is the TFT type. However, the FPD device 35 can be a CMOS type. In the above embodiments, the electronic cassette 13 is portable. However, the X-ray imaging apparatus 2b can be a stationary type installed on the imaging table or floor stand. In the above embodiments, the radiation is X-rays. However, radiographic imaging of the invention may be a type in which gamma rays or the like is used as radiation.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A communication method for a radiographic imaging system including a radiation source for emitting radiation, a radiation source driver for driving said radiation source, and a radiographic imaging apparatus, having a radiographic imaging panel incorporated therein, having pixels arranged for storing signal charge according to said radiation transmitted through an object, for outputting a radiation image of said object by conversion in an electrical signal, wherein a first AEC signal output device is connectable to said radiation source driver, and is discrete from said radiographic imaging apparatus, for detecting a dose of said radiation transmitted through said object to output a first AEC signal for automatic exposure control, said communication method comprising a step of:
carrying out connection in a communication path between said radiographic imaging apparatus and an interface port provided in said radiation source driver for connection with said first AEC signal output device, for transmitting a sync signal for operating said radiographic imaging panel in synchronism with a start of emitting said radiation.

2. A communication method as defined in claim 1, wherein said radiographic imaging apparatus communicates with said interface port with communication compatibility for transmission and reception in relation to a form of said sync signal.

3. A communication method as defined in claim 1, wherein there is communication incompatibility between said interface port and said radiographic imaging apparatus in relation to a form of said sync signal;
said sync signal is converted by a signal converter into a form with communication compatibility.

4. A communication method as defined in claim 3, wherein said signal converter is provided in said radiographic imaging apparatus or in said communication path.

5. A communication method as defined in claim 1, wherein said radiographic imaging apparatus, when said sync signal is transmitted by said communication path with said radiation source driver, changes over said radiographic imaging panel from resetting of unnecessary signal charge of pixels to storing of said signal charge in said pixels.

6. A communication method as defined in claim 1, wherein said radiographic imaging apparatus includes a second AEC signal output device, discrete from said first AEC signal output device, for detecting a dose of said radiation transmitted through said object, to output a second AEC signal for automatic exposure control;
said second AEC signal is transmitted by said communication path to said radiation source driver.

7. A communication method as defined in claim 6, wherein said radiation source driver shuts off irradiation of said radiation for said automatic exposure control according to said second AEC signal.

8. A communication method as defined in claim 7, wherein said second AEC signal output device includes a dose sensor for outputting a dose signal of said dose of said radiation.

9. A communication method as defined in claim 8, wherein said second AEC signal output by said second AEC signal output device is said dose signal;
said radiation source driver includes a judging device for checking whether a cumulative dose of said radiation has reached a target dose according to said dose signal.

10. A communication method as defined in claim 8, wherein said second AEC signal output device is constituted by said dose sensor and a judging device for checking whether a cumulative dose of said radiation has reached a target dose according to said dose signal from said dose sensor;
if reach of said cumulative dose to said target dose is detected by said judging device, a shut-off signal is output for said second AEC signal.

11. A communication method as defined in claim 1, wherein a second sync signal for said radiographic imaging panel in synchronism with shut-off of said radiation is transmitted through said communication path;
said radiographic imaging apparatus, when said second sync signal is transmitted through said communication path with said radiation source driver, changes over said radiographic imaging panel from storing of signal charge in said pixels to reading of said signal charge from said pixels in a signal processing circuit.

12. A communication method as defined in claim 1, wherein said communication path is constituted by a signal line.

13. A communication method as defined in claim 12, wherein said signal line has a signal coupling device with first, second and third contact points;
said first contact point is connected with said interface port;
said second contact point is connected with said radiographic imaging apparatus to constitute said communication path in series with said first contact point;
said third contact point is connected with said first AEC signal output device in a communicable manner with said interface port.

14. A communication method as defined in claim 13, wherein said signal coupling device includes a selector for changing over said second and third contact points and connecting a selected one of said second and third contact points with said first contact point.

15. A communication method as defined in claim 1, wherein said sync signal is transmitted and received wirelessly in said communication path.

16. A radiographic imaging system comprising:
a radiation source for emitting radiation;
a radiation source driver for driving said radiation source;
a radiographic imaging apparatus, having a radiographic imaging panel incorporated therein, having pixels arranged for storing signal charge according to said radiation transmitted through an object, for outputting a radiation image of said object by conversion in an electrical signal;
an interface port, provided in said radiation source driver, for connection with a first AEC signal output device, said first AEC signal output device being discrete from said radiographic imaging apparatus, for detecting a dose of said radiation transmitted through said object to output a first AEC signal for automatic exposure control;
a communication path for connection between said interface port and said radiographic imaging apparatus, and for transmitting a sync signal for operating said radiographic imaging panel in synchronism with a start of emitting said radiation.

17. A radiographic imaging system as defined in claim 16, wherein there is communication incompatibility between said interface port and said radiographic imaging apparatus in relation to a form of said sync signal;
further comprising a signal converter, provided in said communication path, for converting said sync signal into a form with communication compatibility between said interface port and said radiographic imaging apparatus.

18. A radiographic imaging system as defined in claim 16, further comprising a signal coupling device having first, second and third contact points;
wherein said first contact point is connected with said interface port;
said second contact point is connected with said radiographic imaging apparatus to constitute said communication path in series with said first contact point;
said third contact point is connected with said first AEC signal output device in a communicable manner with said interface port.

19. A radiographic imaging system as defined in claim 16, further comprising first and second wireless interfaces, arranged along said communication path, for wirelessly transmitting said sync signal between.

20. A radiographic imaging apparatus for a radiographic imaging system including a radiation source for emitting radiation, a radiation source driver for driving said radiation source, and an interface port, provided in said radiation source driver, for connection with a first AEC signal output device for detecting a dose of said radiation transmitted through said object to output a first AEC signal for automatic exposure control, said radiographic imaging apparatus comprising:
a radiographic imaging panel, discrete from said first AEC signal output device, having pixels arranged for storing signal charge according to said radiation transmitted through said object, for outputting a radiation image of said object by conversion in an electrical signal;
a communication path for connection with said interface port, and for transmitting a sync signal for operating said radiographic imaging panel in synchronism with a start of emitting said radiation;
a controller for operating said radiographic imaging panel according to said sync signal.
